(19) United States
(12) Patent Application Publication
(10) Pub. No.: US 2025/0377722 A1
(43) Pub. Date: Dec. 11, 2025

(54) Ai Andy, Vapor-Based AI Assistant

(71) Applicant: Andy Short, Andover, MA (US)

(72) Inventor: Andy Short, Andover, MA (US)

(21) Appl. No.: 19/223,898

(22) Filed: May 30, 2025

(51) Int. Cl. G06F 3/01; A61M 21/00; A61M 21/02; G02B 30/56; G06F 3/16; G06T 15/08

(52) U.S. Cl. CPC G06F 3/013; A61M 21/02; G02B 30/56; G06F 3/167; G06T 15/08; A61M 2021/0027; A61M 2021/005; G06F 2203/011

(57) ABSTRACT

A tabletop device forms a thin upward-facing fog screen above its housing. Projection optics cast dynamic images onto the screen to create a free-floating avatar. Integrated microphones, speaker, camera and AI control circuitry interpret voice commands and emotional cues, generating synchronized visual and verbal responses for therapeutic and assistive interaction.

### FIELD OF THE INVENTION

This invention relates to interactive artificial intelligence (AI) assistant devices. In particular, it concerns an AI assistant system that uses a vapor-based fog display for visual holographic interaction, combined with sensors and AI algorithms for emotion detection and responsive support. The field of the invention overlaps consumer electronics, human-computer interaction, and therapeutic assistive technology.

### BACKGROUND OF THE INVENTION

AI-powered virtual assistants (e.g., smart speakers and voice assistants) have become common, providing information and home automation via voice. However, conventional assistants lack a physical or visually interactive presence-most are disembodied voices or simple screen-based avatars. This limits user engagement and the sense of companionship. Some advanced systems have explored holographic displays (e.g., “virtual companion” devices), but these often rely on glass screens or projections and do not create a free-standing 3D visual in open air.

Recent developments in fog or vapor projection displays demonstrate that a thin curtain of mist can serve as a floating projection screen, creating images that appear volumetric [symbol] . Fog screens use atomized water vapor as a projection medium, allowing visuals to hover in mid-air like a hologram. This technology can create a “real 3D display effect” and a “comfortable user visual sensory experience” [symbol] . Such displays have not been integrated into personal AI assistant devices so far.

Another aspect is emotional intelligence in AI. Traditional assistants respond to explicit commands but do not deeply gauge the user's emotional state. Research indicates AI chatbots and virtual agents can provide mental health benefits by simulating empathetic listening and therapy techniques [symbol] . For example, a recent trial of an AI therapy chatbot showed significant reductions in depression and anxiety symptoms among users [symbol] . Furthermore, major tech companies are patenting ways for assistants to detect user mood or health from voice and behavior—e.g., Amazon's patent to have Alexa analyze voice for illness or “emotional states” like happiness, sadness, anger, or fear [symbol] . Such context awareness could make AI assistants more supportive, especially in therapeutic or healthcare settings.

Patients in hospitals or individuals in therapy often feel isolated. A friendly AI assistant with a visual presence and emotional awareness could provide comfort, reminders, or company. For instance, a hospital bedside assistant could monitor a patient's mood or detect a cough and notify staff or suggest remedies, similarly to how Alexa's proposed system can detect a cough and suggest soup [symbol] . However, no existing product fully combines: (1) a volumetric fog-based display for an engaging visual persona, (2) multimodal sensors (camera, microphone, etc.) to read user emotions and context, and (3) an AI platform to tailor interactions for applications like mental health support or medical assistance.

Accordingly, there is a need for an AI assistant device that addresses these gaps. The present invention provides an interactive AI assistant called “Ai Andy” that projects an avatar or information onto a vapor fog screen, creating a pseudo-holographic presence. It integrates sensors and AI for emotion detection, enabling empathetic responses. Ai Andy is designed for various use cases, including serving as a therapeutic companion (providing psychological comfort, coaching, or companionship) and as a hospital assistant (monitoring patient cues, answering questions, and alerting caregivers). This invention aims to enhance user engagement and well-being by merging advanced display technology with emotional AI in a standalone assistant device.

### SUMMARY OF THE INVENTION

The invention is an AI assistant device with a vapor-based display and emotion-aware interaction capabilities. The device, referred to as “Ai Andy,” comprises a housing that contains a mist/fog generator, a projection system, audio speakers, a microphone array, cameras, and an AI computing core. The device produces a thin, upwardly-projected fog curtain (a sheet of fine water vapor in air) and projects visual content (such as a virtual avatar or pertinent images/text) onto this fog screen, creating a floating, hologram-like interface.

The AI assistant uses multiple sensors (cameras, microphones, etc.) and algorithms to detect the user's presence, voice commands, and emotional state (e.g., by analyzing vocal tone, facial expressions, or other biometrics). Based on these inputs, the AI can dynamically adjust its responses—for example, adopting a soothing tone and calming visuals if the user appears distressed or sad, or providing more energetic interactions if the user is happy. The assistant thus behaves as a responsive companion rather than a one-size-fits-all voice robot.

Key features and advantages of the invention include:
Vapor-Based Fog Display: A built-in ultrasonic atomizer and air flow system create a stable, laminar mist screen (FIG. 3). A projector (or other light source) projects an image of a virtual character or interface onto the fog, resulting in a free-floating visual that the user can see from the front. This provides a compelling 3D illusion without requiring physical screens or eyewear². The fog screen may be planar or slightly curved, and can be generated on demand during interactions (and turned off when not in use).
Emotion and Context Sensing: Ai Andy is equipped with a high-resolution camera and microphone array to monitor the user's facial expressions, body language, voice tone, and keywords. Using on-device AI models (and/or cloud-based analysis), it can infer user emotions such as happiness, sadness, stress, or anger [symbol] . It also detects context cues (for instance, coughing or sneezing sounds indicating illness, as noted in Amazon's patent [symbol] , or ambient noise indicating location). These capabilities enable more empathetic and context-appropriate responses.
Interactive AI Persona: The device runs an AI assistant software that not only answers questions or executes commands, but also engages the user in dialogue. It can project a friendly avatar (for example, a human-like guide or a comforting abstract animation) on the fog screen (FIG. 1). This avatar's expressions or visuals can change according to the interaction-smiling, showing concern, etc., to mirror and validate the user's feelings. The assistant's voice output (through speakers) likewise adjusts in tone and content, offering encouragement or enthusiasm as needed.
Therapeutic Support Mode: In one use case (FIG. 4), Ai Andy acts as a therapeutic companion. It can conduct guided meditation sessions, prompt users with cognitive-behavioral therapy (CBT) exercises, or simply listen and converse in a supportive manner. For example, if the user is crying or has a depressed tone, the system recognizes this and may respond gently: the avatar might display a calming scene (like a tranquil nature animation) and the AI could say, “I'm here with you. Would you like to talk or do a breathing exercise?” Such tailored support builds on evidence that AI chat agents can alleviate anxiety and depression [symbol] . The device maintains confidentiality and can operate 24/7, supplementing human therapists by offering immediate comfort in moments of distress.
Healthcare Assistant Mode: Another embodiment (FIG. 5) positions Ai Andy at a patient's bedside in a hospital or eldercare facility. The device interfaces with medical data (e.g., it can read basic vital signs via connected sensors or ask the patient about pain levels). It can answer patient questions about their schedule or remind them to take medication. Importantly, using its emotion detection, it might detect if the patient is in pain (from facial cues) or anxious, and automatically notify nursing staff or suggest coping strategies. For instance, a patient appearing uncomfortable could trigger the device to ask, “I see you may be in pain. Should I call a nurse?”—providing proactive care. This addresses gaps in continuous patient monitoring and emotional support. The fog-projected avatar could even be customized (e.g., a friendly animal or character) to cheer up pediatric patients or others.
System Architecture: As shown in FIG. 2, the device's architecture includes a central control unit (CPU/processor 170) running AI software (including speech recognition, natural language understanding, and a dialogue manager with emotional intelligence). A memory 172 stores the AI models and a knowledge base. A communication module 180 (Wi-Fi/Bluetooth) connects to cloud services for heavy AI processing or knowledge updates as needed, though primary functions can run locally for privacy. The sensor suite includes microphones 152 for 360° voice input, a camera 150 (or multiple cameras) for face and gesture tracking, and possibly other sensors (like an IR sensor for presence or a thermal sensor for body temperature). Output components include the projector 140 (which may use DLP or laser technology to project images onto the fog) and speakers 154 for voice/audio. The vapor generator 120 (ultrasonic humidifier) produces micro-fine water droplets that form the fog screen 130 emerging from an opening at the top of the device. The generator has a water reservoir 122 and small fan/airflow system 124 to shape the mist curtain (e.g., a planar upward airflow to keep fog relatively flat and stable). A housing 110 encloses these components in a desktop-friendly form factor (roughly the size of a small lamp or speaker). For safety, the device uses low-voltage components and the mist is cool (no heating element needed, minimizing burn risk).
Sensor and Hardware Layout: FIG. 7 illustrates an example physical layout of Ai Andy's components. The base of the housing 110 contains the water reservoir 122 and atomizer unit 120, with air vents directing the fog upward. Near the top front of the device are the camera 150 (centrally located to face the user) and an array of microphones 152 (placed around the top for far-field voice pickup). The projector 140 is angled to project onto the rising mist approximately 4-8 inches above the device. Speakers 154 (stereo) are mounted on the sides or front to emit the assistant's voice. Status LEDs or a small screen (optional) can be on the base to indicate power, connectivity, or when the device is “listening.” All these components are arranged compactly so that Ai Andy can sit on a desk, bedside table, or countertop (FIG. 1 shows a perspective view on a desk 150). The device is powered via an AC adapter or battery backup.
Operation: In use, when a user wakes the device (by voice trigger or presence detection), the fog screen is activated and the avatar is projected (FIG. 3 shows the fog screen in operation during an interaction). The user's speech is captured by microphones and parsed by the AI. If the user says, for example, “I'm feeling a bit anxious,” the AI system detects the emotional content (either through the words or the tone) and decides an appropriate response. The device might respond verbally with a comforting tone: “I'm sorry you're feeling this way. I can play some calming music or do a breathing exercise with you.” Simultaneously, the avatar's expression might show empathy (soft facial expression) and perhaps a visual breathing guide is animated on the fog screen. FIG. 6 is a flowchart illustrating this emotion detection and response process: at step 602 the system captures user input (voice and image), step 604 analyzes it for emotional cues (using speech sentiment analysis and facial expression recognition), step 606 determines the user's emotional state (e.g., anxiety detected), step 608 selects an appropriate response strategy (such as offering a calming exercise), and step 610 generates the output (both spoken words and visual content). The loop continues as the user responds, creating an interactive dialogue.
Personalization and Learning: Over time, Ai Andy can learn user preferences. It might learn what kind of visualizations or music calms a particular user, or notice patterns (e.g., every evening the user appears sad, so it proactively asks if the user wants to chat or notify a loved one). The AI can adapt the avatar's persona slightly to the user's liking—for instance, using more formal or casual speech as appropriate. All data processing related to emotion can be kept local for privacy, or securely encrypted if sent to cloud for more advanced analysis.

The combination of these features results in an AI assistant that is more engaging, supportive, and context-aware than existing solutions. By using a fog-based pseudo-holographic projection, Ai Andy provides a visual “body” to the AI, enhancing user connection. By incorporating emotional intelligence, it can spot user emotions and states (happy, sad, sick, tired, etc.) [symbol] and respond appropriately-something current mainstream assistants lack. This makes it especially suited for sensitive applications like mental health support and patient care, as the device can both improve outcomes (e.g., reduced anxiety [symbol] ) and escalate concerns when needed (e.g., alert a doctor if a patient's condition seems to worsen, subject to safety protocols).

In summary, Ai Andy—Vapor-Based AI Assistant represents a holistic innovation at the crossroads of AI and display technology. It transforms the user's experience with AI from a disembodied voice to a tangible interactive presence that can see, hear, and feel (in an AI sense) the user's state. This patent application covers the device's structural design, system architecture, and methods of using the device in various scenarios to achieve improved user engagement and well-being.

### BRIEF DESCRIPTION OF THE DRAWINGS

FIG. 1 is a perspective view of the Ai Andy vapor-based AI assistant device (100) shown sitting on a desk (150). The device projects a visual avatar (illustrated as a human figure or interface graphic) on a thin fog screen (130) above its housing, creating a hologram-like presence during user interaction.

FIG. 2 is a schematic block diagram of the system architecture of Ai Andy. It depicts the main functional components, including the control/processing unit (170), memory (172), AI software module, sensor inputs (camera 150, microphones 152, etc.), output devices (projector 140, speakers 154), communication module (180 for network/cloud connectivity), and the fog generation system (120 with reservoir 122 and fan 124).

FIG. 3 is an illustration of the device in operation, showing the fog curtain projection during interaction. It highlights how the mist/fog screen (130) is emitted upward from the device's top and how an image (such as the AI's avatar or information display) is projected onto this fog screen by the projector (140). The user is shown in front of the device engaging with the floating image.

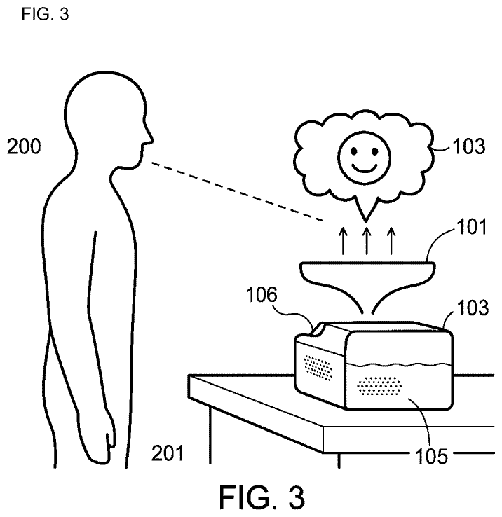

FIG. 4 illustrates a use case scenario where Ai Andy is providing therapeutic support to a user (402). The user appears distressed (e.g., head in hands or sad expression), and the device responds by projecting a calming avatar/scene on the fog screen (130) and speaking comforting words. This figure demonstrates how the device detects the user's emotional state and engages in a supportive dialog (for instance, offering a breathing exercise or empathetic conversation).

FIG. 5 depicts another use case scenario with Ai Andy acting as a hospital assistant for a patient (502) in a medical bed. The device (100) is placed on a bedside table, projecting a gentle avatar on the fog screen (130) and perhaps displaying health-related info. It interfaces with the patient—for example, asking about pain level, providing companionship, or calling a nurse via the hospital network if urgent. Medical equipment (such as an IV stand or monitor 504) is shown nearby to contextualize the environment.

FIG. 6 is a flowchart of the AI emotion detection and response process used by Ai Andy. It outlines the steps: capturing user input (step 602: via microphones/camera), analyzing the input for emotional cues (604), determining the user's emotional or health state (606), selecting an appropriate AI response or action (608), and generating the output (610: e.g., spoken reply, avatar animation, or alert). Conditional decision points illustrate how the system might branch (for instance, if user is detected crying, go down a comforting response path).

FIG. 7 is a diagram of the sensor and hardware layout of the Ai Andy device, in a cutaway or front view. It labels internal and external components: the base housing (110), water vapor generator (120) with its reservoir (122) and nozzle/outlet, the projector unit (140) angled toward the fog area, the primary camera (150) at the top front, multiple microphones (152) around the device for 360° audio pickup, speakers (154) for voice output, and other elements like the processor module (170) and communication unit (180) housed inside. Arrows indicate the flow of vapor and projection. This figure provides a hardware reference for how the compact device is constructed.

### DETAILED DESCRIPTION OF EMBODIMENTS

The following detailed description refers to the accompanying figures to illustrate specific embodiments of the Ai Andy—Vapor-Based AI Assistant. Wherever possible, like reference numbers refer to like elements across the figures for consistency. It is understood that these embodiments are examples, and variations or modifications may be made without departing from the scope of the invention as defined by the claims.

### Device Construction (Physical Embodiment)

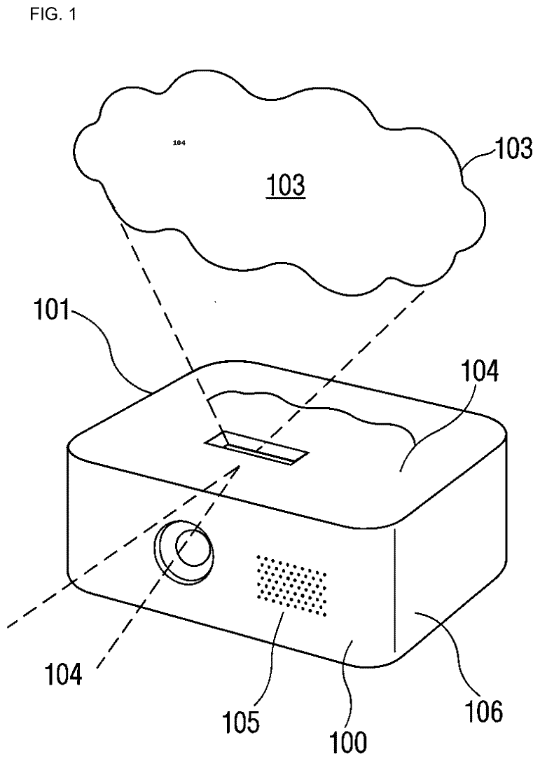

Referring first to FIG. 1, the AI assistant device 100 is shown in a perspective view. The device 100 has an outer housing 110 that is approximately cylindrical or rectangular with a stable base for placement on a surface such as desk 150. In the embodiment shown, the housing 110 is about 8-12 inches tall and contains the internal components. At the top of the housing, an outlet vent 126 is present through which a planar mist or fog screen 130 is produced. As illustrated, a misty, translucent “curtain” of vapor 130 rises upwards from the device, creating a projection surface suspended in the air.

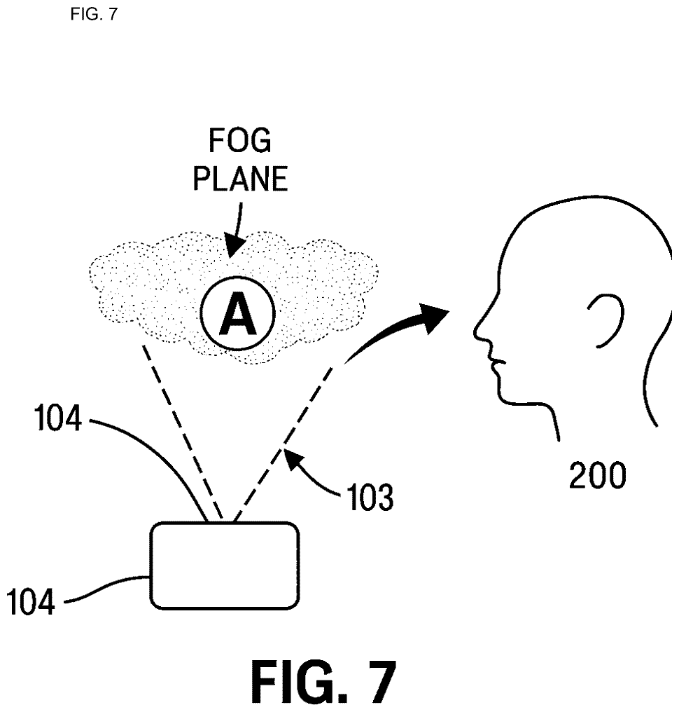

Inside housing 110 (see FIG. 7 for internal layout), a vapor generation module 120 produces this fog curtain 130. The vapor generator 120 preferably includes an ultrasonic atomizer element (a vibrating diaphragm) that ultrasonically agitates water from an internal reservoir 122 into micro-fine droplets (a cool mist). A small fan or air pump 124 directs the mist upward in a laminar flow, forming a thin, relatively stable curtain of fog (approximately 6-8 inches wide, and only a few millimeters thick). This fog screen 130 serves as a dynamic, reconfigurable display screen. Unlike a solid display, it can appear or disappear as needed (the device can swiftly dissipate the fog when turning “off” the display).

Mounted within the upper portion of housing 110 is a projection system 140 (FIG. 7). In one embodiment, projector 140 is a miniature wide-angle projector using LED or laser light sources. It is oriented to project images upward and forward onto the fog screen 130. The focal distance and angle are calibrated so that the image comes into focus precisely on the plane of the fog curtain (which might be, for example, 3-4 inches above the projector lens). The projected image can be a virtual avatar's face or body, text, symbols, or any graphics relevant to the interaction. Because the fog is semi-transparent, the image appears to float in space, and minor movements of the user's head can create a parallax effect enhancing the 3D illusion. (Note: alternative embodiments could use scanning laser projection or other volumetric display techniques, but the fog projection is preferred for its simplicity and human-safe characteristics.)

The device 100 also features multiple sensors to perceive the user and environment. As shown in FIG. 7, a camera 150 is positioned near the top front of the device (just below the fog output area, so it has a clear line of sight to the user). Camera 150 may be a wide-angle RGB camera capable of capturing the user's face and upper body. It feeds images to the AI system for facial expression recognition, identity recognition (to personalize if multiple users), and possibly gesture recognition. In some embodiments, an infrared depth sensor could be added adjacent to camera 150 to improve user tracking in low light or to enable hand-gesture controls via depth mapping of the user's hands.

A microphone array 152 (FIG. 7) is distributed around the device (e.g., 4-6 microphones spaced circularly). These microphones capture the user's voice commands from any direction, enabling far-field speech recognition. The array allows the system to perform noise cancellation and sound source localization (to know which direction the user is speaking from, and to focus on that). The microphones also pick up non-verbal audio cues—e.g., the sound of a cough, crying, laughter, or a stressed tone of voice. This audio data is used by the AI for determining the user's state. For example, a detected cough or sniffle might be interpreted as the user having a cold, consistent with known AI proposals to detect illness via voice [symbol] .

In the embodiment, device 100 includes at least one speaker 154 (or stereo speakers) to output the AI assistant's synthesized speech and other audio (like music or alerts). The speaker is positioned behind a grille on the housing so that sound projects outward. Ai Andy's voice is generated by a text-to-speech engine which can modulate tone and prosody to convey empathy or enthusiasm appropriately.

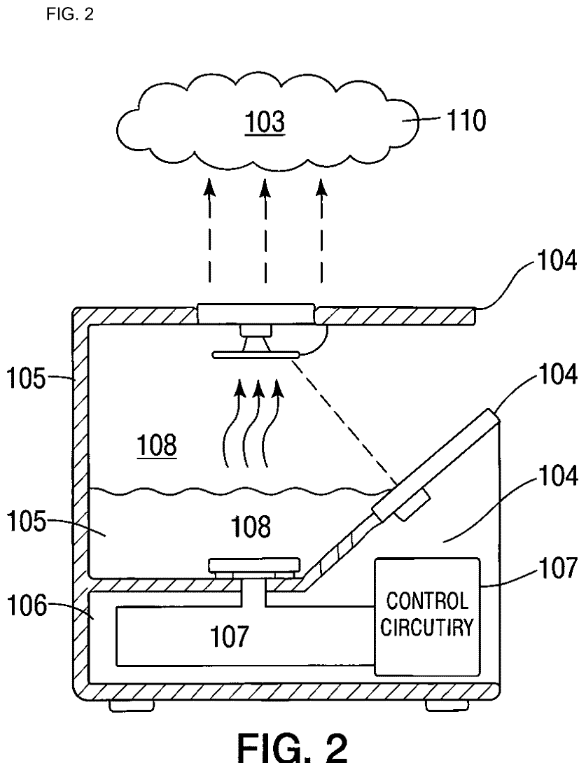

Inside the base of device 100, the control electronics are housed (see FIG. 2 schematic). A processor (CPU) 170 or AI chip runs the core software. This may be an embedded system-on-chip capable of neural network inference for voice and vision analysis. Memory 172 (flash and RAM) stores the programming, including an AI model (or models) for natural language understanding, dialogue management, and emotion recognition. The device can operate in a standalone manner for privacy, but it also has a wireless communication module 180 (such as Wi-Fi and/or LTE) to connect to cloud services if needed—for example, to access an updated knowledge database, perform heavy language processing tasks on a server, or download software updates. The communication also allows integration into external systems (e.g., a hospital network or smart home system).

The device 100 is powered by an AC adapter. Optionally, a battery backup may be included for short-term portable use or to safely shut down in a power outage. A power management circuit ensures stable power to the projector 140, which is sensitive to voltage changes.

The exterior design of housing 110 can vary. In one aesthetic embodiment, it has a minimalist smooth cylindrical shape with a subtle indicator light on front. The fog emerges from a slot-like vent on top. Another embodiment might have a slight anthropomorphic design (e.g., a subtle suggestion of shoulders or a head shape in the device form) to psychologically reinforce the presence of the avatar. These design variations do not alter the core function.

### System and Functional Operation:

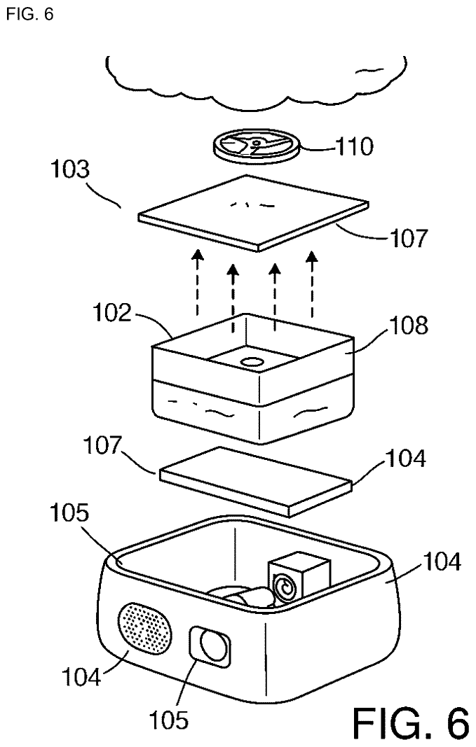

Now referring to FIG. 2 (system block diagram) in conjunction with FIG. 6 (flowchart), the operation of Ai Andy's AI system will be described. When in standby, the device 100 monitors its environment for a “wake word” (like “Andy” or a custom name) using the microphone array 152 and on-device keyword spotting. It may also periodically use camera 150 to detect if a person is present nearby. In standby, the fog generator is off (to conserve water and avoid unnecessary mist).

Wake and Display Activation: When a user says the wake word or presses an optional wake button, the control unit 170 transitions the device to active mode. The fog generator 120 is activated, producing the fog screen 130 (typically reaching full projection thickness in under 2 seconds). The projector 140 turns on and displays a welcome animation or the avatar's “idle” state on the fog (for instance, a gently pulsating icon or a friendly face saying “Hello”). The speakers 154 play a greeting or chime. This immediate visual feedback is important to signal to the user that the device is ready and “listening.”

Input Capture and Analysis (FIG. 6, step 602 & 604): The user can speak naturally to Ai Andy. The microphone array 152 captures the speech audio. The AI's speech recognition module converts the audio to text in real time. Simultaneously, the raw audio is analyzed for paralinguistic features—volume, pitch, pace, tone. This allows the system to detect, for example, if the voice is shaky (maybe user is upset) or unusually loud (user might be angry or in a panic). The camera 150 captures the user's face; a face analysis algorithm computes facial landmarks and expressions (smile frown, eye gaze, etc.). For example, it might detect reddened eyes and downturned lips indicating sadness, or wide eyes indicating surprise. These data (from voice and face) feed into an Emotion Inference Engine (an AI model) which estimates the user's likely emotional state (FIG. 6, step 606). The states could be labeled categories (happy, neutral, sad, angry, fearful, etc.) or a dimensional value (e.g., a stress level from 0 to 100). Known techniques from affective computing are applied here to give the assistant a sense of the user's mood [symbol] . If the confidence in detection is low or ambiguous, the system can remain in a neutral response mode to avoid mistakes.

Natural Language Understanding: In parallel, the recognized text of the user's speech is processed by the AI's natural language understanding (NLU) module to determine the user's intent or query. For example, the user might ask a factual question (“What's the weather?”), express a feeling (“I had a hard day”), or issue a command (“Remind me to take my meds at 8 PM”). The context (like time of day, whether this user has asked similar questions before) is also considered. The Emotional state from the previous step (if available) provides additional context—e.g., the same request “How was my last therapy session?” would be handled differently if the user is detected as sad versus cheerful.

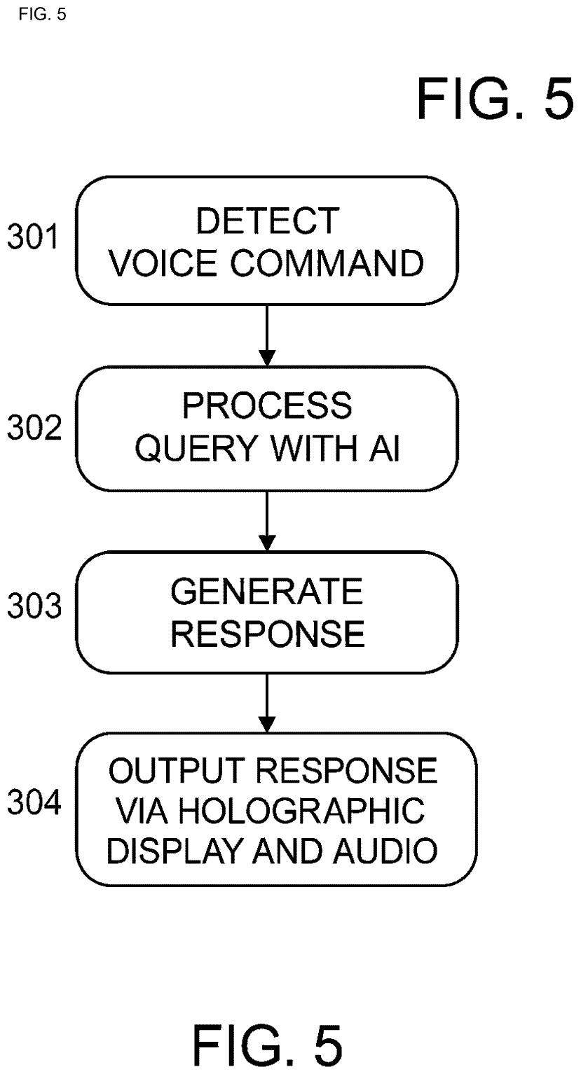

Dialogue Management and Response Selection (FIG. 6, step 608): The Dialogue Manager in the AI core (170) uses the understood intent plus emotional context to formulate an appropriate response. This may involve multiple components: a standard transactional response (answering a question, performing an action) and an empathetic layer. For instance, if the user says in a depressed tone “I feel so overwhelmed,” the dialogue manager recognizes this as an emotional statement rather than a factual query. It could choose a therapeutic response strategy: perhaps asking a gentle follow-up (“I'm sorry to hear that. Do you want to talk about what's overwhelming you?”) or suggesting a coping activity. If the user asked a factual question but is detected as sad, the system might answer the question and then kindly ask, “By the way, you seem a bit down-I'm here if you need anything.” These nuanced responses distinguish Ai Andy from a typical assistant. The system's response selection logic may reference programmed rules (for critical cases like if user says something indicating possible self-harm, it might suggest contacting a professional or calling a predefined emergency contact) or machine-learned conversation models. In a hospital setting (FIG. 5 scenario), if a patient asks “When is my next dose of medicine?” in a pained voice, the system can answer with the scheduled time and also ask if they need help from a nurse, since pain is apparent.

Output Generation (FIG. 6, step 610): Once the content of the response is decided, the system generates the multi-modal output. The text of the reply is sent to the text-to-speech engine, which produces spoken audio. The voice is chosen to be friendly and can vary its inflection—e.g., a softer, slower tone if the user is upset (the system might literally lower the pitch/volume of the synthetic voice to sound soothing). At the same time, the Visual Renderer creates appropriate imagery for the fog screen. If an avatar character is being used, it will lip-sync to the spoken words and display a matching facial expression (e.g., a concerned look for empathy, a smile for positive feedback). The projector 140 displays this animated avatar on fog 130. Alternatively or additionally, the system might show relevant visual content: for a therapy exercise, maybe a simple breathing guide (a circle expanding and contracting to guide inhale/exhale); for a factual answer, maybe text or an icon; for a joke or mood-lifting attempt, perhaps a funny animation. The coordination of audio and visual output provides a rich, engaging interaction.

Continuous Interaction and Adaptation: The interaction continues turn by turn. The user responds, the system listens and analyzes again (looping back to step 602). The fog display 130 can remain on as long as the session continues. If the user stops interacting for a certain time (say, no speech or user walks away), the system can politely say it will be on standby and then turn off the projection (fog dissipates) until needed again. The device's software learns over time. For example, it might note that a particular user responds better to humor when sad, so it uses light humor more after detecting sadness in that user in the future, personalizing the approach. Machine learning models for personalization can run locally or in the cloud (with user permission).

Safety and Privacy Considerations: The device is designed with privacy in mind. All audio and video processing for emotion detection can be done on-device; nothing needs to be uploaded unless the user opts into cloud services for advanced features. The camera 150 can have a physical shutter or indicator LED to assure users when it's active. In a hospital context, the device complies with privacy regulations (e.g., HIPAA) by storing or transmitting any personal data in encrypted form. The water used for fog is contained and minimal; sensors detect if the device is tilted or if water is low, and it can safely shut off to prevent spills. The fog itself is room-temperature and poses no burn or respiratory risk (it's similar to a cool-mist humidifier).

### Example Scenarios:

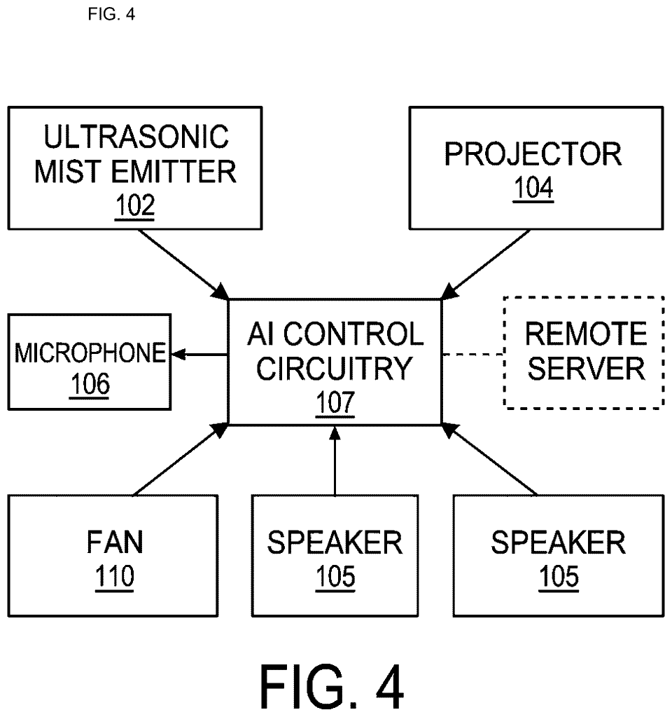

Therapy Companion (FIG. 4): A user (402) is at home after a stressful day. They trigger Ai Andy and say, “I don't know what to do, I just feel really anxious.” The camera 150 sees their slumped posture, the microphone 152 picks up a quiver in their voice. Ai Andy's emotion engine classifies this as high anxiety. The device's avatar appears with a concerned expression. It speaks softly: “I'm sorry you're feeling like this. Let's try a short breathing exercise together.” On the fog screen 130, a visual of a blue circle expanding and contracting appears, and the voice guides the user to breathe in sync. After a couple of minutes, the assistant gently asks how the user feels now. This continues, potentially moving into a conversation about the user's day, employing evidence-based therapeutic prompts. Over several weeks of use, the user forms a habit of “talking to Ai Andy” each evening, and the system's supportive responses contribute to measurable improvements in the user's mood and resilience (as supported by research in AI therapy bots [symbol] ).
Hospital Assistant (FIG. 5): A patient (502) recovering from surgery has Ai Andy on the bedside. At 2 AM, the patient wakes up and groans in pain. The device's microphone picks up the groan and increased breathing rate; camera notes the patient's facial wince. The system recognizes signs of pain and speaks: “I notice you seem uncomfortable. Should I call a nurse for you?” The patient says yes. Ai Andy signals the nurse station or sends a notification through the hospital system (via module 180). While waiting, the device calmly chats to reassure the patient (“A nurse is on the way. Try to take slow breaths. You're doing great.”). The fog screen might display a simple calming animation (like a dim, soothing light pattern) rather than the avatar to avoid overstimulation in the dark room. In another scenario, the patient could ask, “What's my schedule today?” The device, having access (with permission) to the patient's appointment schedule, projects a list (e.g., “Physical therapy at 10:00, Doctor round at 1:00”) and reads it aloud. The device could also remind the patient to do prescribed exercises or take medication, enhancing adherence to care plans. Nurses and doctors can review Ai Andy's logs to see if the patient requested help or how often they reported pain, providing valuable data for care.

These scenarios demonstrate the flexibility of the invention: it can function as a general smart assistant, but crucially it adds the dimensions of visual presence and emotional intelligence. The fog-based display captivates users and reduces the sense of interacting with a machine—it feels more like a companion is present. The emotional/context awareness allows it to play roles that require empathy and understanding, which is a leap beyond current AI assistant capabilities.

### ALTERNATIVES AND EXTENSIONS

While the above description focuses on a particular embodiment, the invention can be varied. For instance, the form factor could be made portable (a smaller device with a shorter fog plume, or even a wearable pendant that projects a tiny image). The fog projection could be extended to 360° visibility by projecting on a mist that flows upward in a cylindrical shape (using multiple projectors)—enabling multiple people to view the avatar around a table, for example. The avatar itself could be user-customizable (perhaps the user chooses a character or even loads a custom face to be projected, such as a familiar cartoon or a friendly abstract shape, depending on preference). The AI software could integrate with third-party health or wellness services (for example, syncing with a user's fitness tracker to know if they had poor sleep, so it adjusts its interactions accordingly).

Additionally, although the described use cases are therapy and hospital, Ai Andy could be used in educational settings (a tutor that senses if a student is frustrated and alters its teaching approach) or customer service (a concierge device in a hotel lobby with a visually welcoming presence). The patent thus encompasses any application where a vapor display AI assistant enhances user interaction by combining visual, auditory, and emotional channels.

Construction materials and specifics can also vary: e.g., the water reservoir 122 could be refillable or accept cartridges; the fog could be generated by alternative methods (compressed air+liquid nozzles, etc.) as long as it results in a similar projection medium. The term “fog screen” here includes any flowing particulate medium suitable for image projection [symbol] (e.g., water vapor, dry ice mist, or even fine dust if moisture must be avoided, though water is preferred for safety and ease).

The descriptions of the figures and embodiments above are intended to be illustrative and not limiting. The scope of the invention is defined by the following claims.

1. A vapor-based holographic display apparatus comprising:
(a) a housing;
(b) an ultrasonic mist emitter positioned in the housing and configured to generate a fog screen above the housing;
(c) a projector oriented to project an image onto the fog screen, thereby forming a visible avatar;
(d) at least one microphone configured to receive a user voice command;
(e) at least one speaker configured to output audio; and
(f) control circuitry operatively coupled to the mist emitter, projector, microphone and speaker, the control circuitry programmed to
process the voice command using an artificial intelligence model and, in response, drive the projector and speaker to produce a synchronized visual and verbal reply.

2. The apparatus of claim 1 wherein the control circuitry determines an emotional state of the user from the voice command and selects the verbal reply based on the emotional state.

3. The apparatus of claim 1 wherein the projector comprises a laser scanning engine.

4. The apparatus of claim 1 further comprising a fan configured to direct the fog screen upward.

5. The apparatus of claim 4 wherein fan speed is modulated by the control circuitry to stabilize the fog screen.

6. The apparatus of claim 1 further comprising a camera disposed on the housing above the projector.

7. The apparatus of claim 6 wherein the control circuitry tracks user gaze using input from the camera.

8. The apparatus of claim 1 wherein the control circuitry communicates with a remote server via a wireless transceiver.

9. The apparatus of claim 1 wherein the ultrasonic mist emitter operates at a frequency of at least 1.7 MHz.

10. The apparatus of claim 1 wherein the fog screen has a thickness no greater than 3 mm.

11. The apparatus of claim 1 wherein the avatar includes facial expressions selected in response to the emotional state.

12. The apparatus of claim 1 wherein a portion of the artificial intelligence model executes on the remote server.
